(19) United States
(12) Patent Application Publication
(10) Pub. No.: US 2010/0004313 A1
(43) Pub. Date: Jan. 7, 2010

(54) Modified Poloxamers for Gene Expression and Associated Methods

(75) Inventors: Gregory Slobodkin, Huntsville, AL (US); Majed Matar, Madison, AL (US); Brian Jeffery Sparks, Huntsville, AL (US); Jason Fewell, Madison, AL (US); Khursheed Anwer, Madison, AL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: TBD

(21) Appl. No.: 12/395,240

(22) Filed: Feb. 27, 2009

Related U.S. Application Data
(60) Provisional application No. 61/067,607, filed on Feb. 29, 2008.

(51) Int. Cl. A61K 48/00; C08G 65/00

(52) U.S. Cl. 514/44 A; 525/409; 514/44 R

(57) ABSTRACT

Nucleotide delivery polymers, compositions, and associated methods for the enhancement of gene delivery and expression in solid tissues are provided. In one aspect, for example, a nucleotide delivery polymer may include a poloxamer backbone having a metal chelator covalently coupled to at least one terminal end of the poloxamer backbone. In another aspect, the nucleotide expression polymer has a metal chelator coupled to at least two terminal ends of the poloxamer backbone.

### CROSS-REFERENCE TO RELATED APPLICATIONS

This application claims the benefit of Provisional Application No. 61/067,607, filed Feb. 29, 2008, which is incorporated herein by reference in its entirety.

### FIELD OF THE INVENTION

The present invention relates to compositions and methods for delivering nucleic acids to solid tissues. Accordingly, this invention involves the fields of molecular biology and biochemistry.

### DESCRIPTION OF THE RELATED ART

Synthetic gene delivery vectors have considerable advantage over viral vectors due to better safety compliance, simple chemistry, and cost-effective manufacturing. However, the use of synthetic gene delivery vectors has been hampered by problems associated with low transfection efficiency as compared to that of the viral vectors. It is believed that intra- and extracellular degradation of nucleic acid sequences may be one of the major contributors to the low transfection efficiencies observed. Aqueous suspensions of DNA complexes with synthetic vectors appear to be generally unstable and aggregate over time, especially at concentrations required for optimal dosing in a clinical setting. This physical instability may also contribute to the loss of transfection activity. Manifestation of particle rupture or fusion due to high curvature of the lipid bilayer or physical dissociation of lipid from DNA have also been postulated as potential underlying reasons for poor stability and aggregation of cationic lipid based gene delivery complexes. Chemical modification such as oxidative hydrolysis of the delivery vectors may also contribute to particle instability.

### SUMMARY OF THE INVENTION

The present invention provides nucleotide delivery polymers, compositions, and associated methods for the enhancement of nucleotide sequence delivery and or expression in solid tissues and body cavities.

In one aspect, the invention provides compounds of formula I:

R^{A}—O—A-B-C-R^{C} (I)

and pharmaceutically acceptable salts thereof, wherein,
A is (—C₂H₄—O—)₂₋₁₄₁;
B is (—C₃H₆—O—)₁₆₋₆₇;
C is (—C₂H₄—O—)₂₋₁₄₁;
R^{A} and R^{C} are the same or different, and are R′-L- or H, wherein at least one of R^{A} and R^{C} is R′-L-;
L is a bond, —CO—, —CH₂—O—, or —O—CO—;
R′ is a metal chelator, wherein the metal chelator is R^{N}NH—, R^{N}₂N—, (R″-(N(R″)—CH₂CH₂)ₓ)₂-N—CH₂CO—, a crown ether selected from the group consisting of 12-crown-4, 15-crown-5, 18-crown-6, 20-crown-6, 21-crown-7, or 24-crown-8,
   wherein the crown ether may have one or more of the crown ether oxygens independently replaced by NH or S, one or more of the crown ether —CH₂—CH₂—replaced by —C₆H₄—, —C₁₀H₆—, or —C₆H₁₀—, one or more of the crown ether —CH₂—O—CH₂—replaced by —C₄H₂O— or —C₅H₃N—, or any combination thereof,
   a cryptand selected from the group consisting of (1,2,2) cryptand, (2,2,2) cryptand, (2,2,3) cryptand, or (2,3,3) cryptand,
   wherein the cryptand may have one or more of the cryptand ether oxygens independently replaced by NH or S, one or more of the crown ether —CH₂—CH₂— moieties replaced by —C₆H₄—, —C₁₀H₆—, or —C₆H₁₀—, one or more of the crown ether —CH₂—O—CH₂— moieties replaced by —C₄H₂O— or —C₅H₃N—, or any combination thereof;
each R^{N} is independently H-(R^{D})₁₋₅, wherein each R^{D} is independently —NH(CH₂CH₂)—, —NH(CH₂CH₂CH₂)—, or —NH (CH₂CH₂CH₂CH₂)—;
each x is independently 0-2;

### and R″ is HO₂C—CH₂—.

In another aspect, for example, a nucleotide delivery polymer may include a poloxamer backbone having a metal chelator covalently coupled to at least one terminal end of the poloxamer backbone. In another aspect, the nucleotide delivery polymer has a metal chelator coupled to at least two terminal ends of the poloxamer backbone. In yet another aspect, a metal chelator may be included in the composition as a coformulant, and thus would not be covalently attached to the poloxamer backbone.

Various metal chelators may be utilized in various aspects of the present invention. In one aspect, for example, the metal chelator may be a cyclic metal chelator. In one specific aspect, such a cyclic metal chelator may include crown ethers, substituted-crown ethers, cryptands, substituted-cryptan, and combinations thereof.

In another aspect, the metal chelator may be an open chain metal chelator. In one specific aspect, such an open chain metal chelator may include EDTA, DTPA, and combinations thereof. In another specific aspect, the open chain metal chelator may be a short polyamine metal chelator.

In another aspect, the present invention provides a nucleotide expression composition including a nucleotide sequence, and a poloxamer backbone having a metal chelator covalently coupled to at least one terminal end of the poloxamer backbone, and wherein the nucleotide sequence is associated with the poloxamer backbone.

Numerous nucleotide sequences are contemplated, including non-limiting examples such as DNA, RNA, siRNA, RNAi, mRNA, shRNA, microRNA, and combinations thereof. Additionally, in one aspect the nucleotide sequence is a plasmid encoding for at least one of RNAi, siRNA, shRNA, microRNA, and mRNA. In another aspect, the nucleotide sequence is a plasmid encoding for a peptide. Specific non-limiting examples of peptides may include interleukin-2, interleukin-4, interleukin-7, interleukin-12, interleukin-15, interferon-α, interferon-β, interferon-γ, colony stimulating factor, granulocyte-macrophage colony stimulating factor, angiogenic agents, clotting factors, hypoglycemic agents, apoptosis factors, anti-angiogenic agents, thymidine kinase, p53, IP10, p16, TNF-α, Fas-ligand, tumor antigens, neuropeptides, viral antigens, bacterial antigens, and combinations thereof. In yet another aspect, the nucleotide sequence is an anti-sense molecule configured to inhibit expression of a therapeutic peptide. In a further aspect, the nucleotide sequence is a siRNA and the metal chelator is a crown ether.

The present invention additionally provides methods for using polymeric vehicles and compositions. In one aspect, for example, a method of enhancing delivery and/or expression of a nucleotide sequence in a solid tissue of a subject may include mixing the nucleotide sequence with a nucleotide delivery polymer to form a nucleotide delivery composition, the nucleotide expression polymer further comprising a poloxamer backbone having a metal chelator covalently coupled to at least one terminal end of the poloxamer backbone. The method may further include delivering the nucleotide expression composition into the solid tissue of the subject. In one aspect, the solid tissue may include solid tumors, muscle tissue, fat tissue, connective tissue, joint tissue, neural tissue, organ tissue, bone tissue, skin tissue, and combinations thereof.

In another aspect, the invention provides methods for enhancing delivery and/or expression of a nucleotide sequence within at least one body cavity of a mammal, preferably a human.

In still another aspect, the invention provides compounds of the formula:

R^{A}—O—pol-R^{C}

and pharmaceutically acceptable salts thereof, wherein pol represents

(a)-poly (—C₂H₄—O—)-poly (—C₃H₆—O-)-poly (—C₂H₄—O—)—or

(b)-poly (—C₃H₆—O-)-poly (—C₂H₄—O—)-poly (—C₃H₆—O-)-;
R^{A} and R^{C} are the same or different, and are R′-L- or H, wherein at least one of R^{A} and R^{C} is R′-L-;
L is a bond, —CO—, —CH₂—O—, or —O—CO—; and
R′ is a cyclic metal chelator or an open chain metal chelator.

### DESCRIPTION OF THE DRAWINGS

FIGS. 1A and 1B show results of electrophoresis of DNA formulated with compounds of the invention at various concentrations.

FIG. 2 is a graph showing SeAP expression levels in mouse serum following i.m. treatment with a SeAP formulated with a compound of the invention.

FIG. 3 is a graph showing SeAP expression levels in mouse serum following i.m. treatment with a SeAP formulated with a compound of the invention.

FIG. 4 shows a graph of hSeAP levels after intra-articular injection of unformulated hSeAP and hSeAP formulated with a compound of the invention into the knees of female ICR mice.

FIG. 5 is a graph showing survival of syngenic CH3 mice following administration of 5×10⁵ murine squamous cell carcinoma VII (SCCVII) cells and subsequent treatment with mouse IL-12 plasmid (pmIL-12) formulated with a compound of the invention.

FIG. 6 is a graph showing gene expression in tibialis muscle of ICR mice after administration of siRNA targeting MMP2 formulated with a compound of the invention.

### DETAILED DESCRIPTION OF THE INVENTION

A preferred class of compounds of formula I are compounds of formula I-a having an open chain metal chelator and pharmaceutically acceptable salts thereof. Compounds of Formula I-a are those wherein,
A is (—C₂H₄—O—)₁₂₋₁₄₁;
B is (—C₃H₆—O—)₂₀₋₅₆;
C is (—C₂H₄—O—)₁₂₋₁₄₁;
R^{A} and R^{C} are the same or different, and are R′-L- or H, wherein at least one of R^{A} and R^{C} is R′-L-;
L is a bond, —CO—, —CH₂—O—, or —O—CO—;
R′ is a metal chelator, wherein the metal chelator is R^{N}NH—, R^{N}₂N—, (R″-(N(R″)—CH₂CH₂)ₓ)₂-N—CH₂CO—,
each R^{N} is independently H-(R^{D})₁₋₅, wherein each R^{D} is independently —NH(CH₂CH₂)—, —NH(CH₂CH₂CH₂)—, or —NH (CH₂CH₂CH₂CH₂)—;
   each x is independently 0-2;

### and R″ is HO₂C—CH₂—.

Preferred compounds of formula I-a are compounds of I-b, wherein, R^{A} is R′-L-; R^{C} is H; L is —CO—; R′ is R^{N}NH—; and

R^{N} is H-(R^{D})₁₋₅, wherein each R^{D} is independently NH(CH₂CH₂)—, —NH(CH₂CH₂CH₂)—, or NH(CH₂CH₂CH₂CH₂)—.

Preferred compounds of formula I-b are compounds of I-c, wherein, R′ is

### NHCH₂CH₂CH₂CH₂NHCH₂CH₂CH₂NH₂,

### —NHCH₂CH₂CH₂CH₂NHCH₂CH₂CH₂CH₂NH₂, or

### N(CH₂CH₂CH₂CH₂NH₂) (CH₂CH₂CH₂NH₂).

Other preferred compounds of formula I-a are compounds of I-d, wherein, R^{A} and R^{C} are the same or different, and are R′-L-; L is —CO—; R′ is R^{N}₂N—; and each R^{N} is independently H-(R^{D})₁₋₅, wherein each R^{D} is independently —NH(CH₂CH₂)—, —NH(CH₂CH₂CH₂)—, or —NH(CH₂CH₂CH₂CH₂)—

Preferred compounds of formula I-d are compounds of I-e, wherein, each R′ is

independently —NHCH₂CH₂CH₂CH₂NHCH₂CH₂CH₂NH₂, —NHCH₂CH₂CH₂CH₂NHCH₂CH₂CH₂CH₂NH₂, or —N(CH₂CH₂CH₂CH₂NH₂) (CH₂CH₂CH₂NH₂) .

Still more preferred compounds of formula I-a are compounds of I-f, wherein, R^{A} is R′-L-; R^{C} is H; L is a bond; and R′ is R″₂-N—CH₂CO—, R″₂N—CH₂CH₂—N (R″)—CH₂CO—, (R″₂N—CH₂CH₂)₂—N—CH₂CO—, or R″₂N—CH₂CH₂—N (R″)—CH₂CH₂—N (R″)—CH₂CO—.

Another preferred class of compounds of formula A are compounds (II-a) having a cyclic metal chelator and pharmaceutically acceptable salts thereof. The invention provides compounds wherein,
A is (—C₂H₄—O—)₁₂₋₁₄₁;
B is (—C₃H₆—O—)₂₀₋₅₆;
C is (—C₂H₄—O—)₁₂₋₁₄₁;
R^{A} and R^{C} are the same or different, and are R′-L- or H, wherein at least one of R^{A} and R^{C} is R′-L-;
L is a bond, —CO—, —CH₂—O—, or —O—CO—; and
R′ is a metal chelator, wherein the metal chelator is a crown ether selected from the group consisting of 12-crown-4, 15-crown-5, 18-crown-6, 20-crown-6, 21-crown-7, or 24-crown-8,
   wherein the crown ether may have one or more of the crown ether oxygens independently replaced by NH or S, one or more of the crown ether —CH₂—CH₂—replaced by —C₆H₄—, —C₁₀H₆—, or —C₆H₁₀—, one or more of the crown ether —CH₂—O—CH₂—replaced by —C₄H₂O— or —C₅H₃N—, or any combination thereof,
   a cryptand, selected from the group consisting of (1,2,2) cryptand, (2,2,2) cryptand, (2,2,3) cryptand, or (2,3,3) cryptand,
wherein the cryptand may have one or more of the cryptand ether oxygens independently replaced by NH or S, one or more of the crown ether —CH₂—CH₂— moieties replaced by —C₆H₄—, —C₁₀H₆—, or —C₆H₁₀—, one or more of the crown ether —CH₂—O—CH₂— moieties replaced by —C₄H₂O— or —C₅H₃N—, or any combination thereof.

Preferred compounds of formula II-a are compounds of II-b, wherein, L is —CH₂—O—or —CO—; and each R′ is independently a cyclic metal chelator, wherein the metal chelator is a crown ether selected from the group consisting of 12-crown-4, 15-crown-5, 18-crown-6, 20-crown-6, 21-crown-7, or 24-crown-8, wherein the crown ether may have one or more of the cryptan ether oxygens independently replaced by NH or S, one or more of the crown ether —CH₂—CH₂— moieties replaced by —C₆H₄—, —C₁₀H₆—, or —C₆H₁₀—, or one or more of the crown ether —CH₂—O—CH₂—moieties replaced by —C₄H₂O— or —C₅H₃N—, or any combination thereof.

Preferred compounds of formula II-b are compounds of II-b, wherein, L is —CH₂—O—; and each R′ is independently a crown ether selected from the group consisting of 12-crown-4, 15-crown-5, 18-crown-6, 20-crown-6, 21-crown-7, or 24-crown-8.

Other preferred compounds of formula II-a are compounds of II-c, wherein, L is —CH₂—O—or —CO—; and each R′ is independently a crown ether selected from the group consisting of 12-crown-4, 15-crown-5, 18-crown-6, 20-crown-6, 21-crown-7, or 24-crown-8, wherein the crown ether has one or more of the crown ether oxygens independently replaced by NH or S.

Preferred compounds of formula II-c are compounds of II-d, wherein, L is —CH₂—O—or —CO—; and each R′ is independently a crown ether selected from the group consisting of 12-crown-4, 15-crown-5, 18-crown-6, 20-crown-6, 21-crown-7, or 24-crown-8, wherein all of the crown ether oxygens are replaced by NH.

Other preferred compounds of formula II-a are compounds of II-e, wherein, L is —CH₂—O—or —CO—; and each R′ is independently a crown ether selected from the group consisting of 12-crown-4, 15-crown-5, 18-crown-6, 20-crown-6, 21-crown-7, or 24-crown-8, wherein one or more of the crown ether —CH₂—CH₂— moieties is replaced by —C₆H₄—, —C₁₀H₆—, or —C₆H₁₀—, or one or more of the crown ether —CH₂—O—CH₂— moieties is replaced by —C₄H₂O— or —C₅H₃N—.

Preferred compounds of formula II-e are compounds of II-f, wherein one or more of the crown ether —CH₂—CH₂— moieties is replaced by —C₆H₄—, —C₁₀H₆—, or —C₆H₁₀—.

More preferred compounds of formula II-f are compounds of II-g, wherein one or two of the crown ether —CH₂—CH₂— moieties is replaced by —C₆H₄—.

Preferred compounds of formula II-e are compounds of II-h, wherein one or more of the crown ether —CH₂—O-CH₂— moieties is replaced by —C₄H₂O— or —C₅H₃N—.

Preferred compounds of formula II-a are compounds of II-i, wherein, L is —CH₂—O—or —CO—; and each R′ is independently a cyclic metal chelator, wherein the metal chelator is a crown ether selected from the group consisting of 12-crown-4, 15-crown-5, 18-crown-6, 20-crown-6, 21-crown-7, or 24-crown-8, wherein the crown ether may have one or more of the crown ether oxygens independently replaced by NH or S, one or more of the crown ether —CH₂—CH₂— moieties replaced by —C₆H₄—, —C₁₀H₆—, or —C₆H₁₀—, or one or more of the crown ether —CH₂—O—CH₂—moieties replaced by —C₄H₂O— or —C₅H₃N—, or any combination thereof.

Preferred compounds of formula II-i are compounds of II-j, wherein, L is —CH₂—O—; and each R′ is independently a crown ether selected from the group consisting of 12-crown-4, 15-crown-5, 18-crown-6, 20-crown-6, 21-crown-7, or 24-crown-8.

Other preferred compounds of formula II-i are compounds of II-k, wherein, L is —CH₂—O—or —CO—; and each R′ is independently a crown ether selected from the group consisting of 12-crown-4, 15-crown-5, 18-crown-6, 20-crown-6, 21-crown-7, or 24-crown-8, wherein the crown ether has one or more of the crown ether oxygens independently replaced by NH or S.

Preferred compounds of formula II-k are compounds of II-1, wherein, L is —CH₂—O—or —CO—; and each R′ is independently a crown ether selected from the group consisting of 12-crown-4, 15-crown-5, 18-crown-6, 20-crown-6, 21-crown-7, or 24-crown-8, wherein all of the crown ether oxygens are replaced by NH.

Other preferred compounds of formula II-i are compounds of II-m, wherein, L is —CH₂—O—or —CO—; and each R′ is independently a crown ether selected from the group consisting of 12-crown-4, 15-crown-5, 18-crown-6, 20-crown-6, 21-crown-7, or 24-crown-8, wherein one or more of the crown ether —CH₂—CH₂— moieties is replaced by —C₆H₄—, —C₁₀H₆—, or —C₆H₁₀—, or one or more of the crown ether —CH₂—O—CH₂— moieties is replaced by —C₄H₂O— or —C₅H₃N—.

Preferred compounds of formula II-m are compounds of II-n, wherein one or more of the crown ether —CH₂—CH₂— moieties is replaced by —C₆H₄—, —C₁₀H₆—, or —C₆H₁₀—.

More preferred compounds of formula II-n are compounds of II-o, wherein one or two of the crown ether —CH₂—CH₂— moieties is replaced by —C₆H₄—.

Preferred compounds of formula II-m are compounds of II-h, wherein one or more of the crown ether —CH₂—O-CH₂— moieties is replaced by —C₄H₂O— or —C₅H₃N—.

It is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to a polymer containing “a molecule” includes reference to a polymer having one or more of such molecules, and reference to “an antibody” includes reference to one or more of such antibodies.

As used herein, the term poloxamer refers to molecules having the general formula HO—(C₂H₄O)ₐ(C₃H₆O)_{b}(C₂H₄O)_{c}—H in which a and c are approximately equal. See, Handbook of Biodegradable Polymers, Chapter 12′ “The Poloxamers: Their Chemistry and Medical Applications” authored by Lorraine E. Reeve. Because the poloxamers are the products of a sequential series of reactions, the chain lengths of individual poloxamer blocks are statistical distributions about the average chain length. Thus, in Formula I, the number of ethyleneoxy groups within A and C and the number of propylenoxy groups within B are meant to be averages.

The meroxapols are block polymers of the following general formula: PPO-EO-PPO. The meroxapols can be represented by the formula HO-poly(C₃H₆O)-poly(C₂H₄O)-poly(C₃H₆O)—H, where PPO and EO refer to polypropyleneoxy and polyethyleneoxy units respectively. The terminal hydroxy groups on these polymers are secondary hydroxy groups.

As used herein, the terms “transfecting” and “transfection” refer to the transportation of nucleic acids from the environment external to a cell to the internal cellular environment, with particular reference to the cytoplasm and/or cell nucleus. Without being bound by any particular theory, it is to be understood that nucleic acids may be delivered to cells either after being encapsulated within or adhering to polymer complexes or being entrained therewith. Particular transfecting instances deliver a nucleic acid to a cell nucleus.

As used herein, “subject” refers to a mammal that may benefit from the administration of a drug composition or method of this invention. Examples of subjects include humans, and may also include other animals such as horses, pigs, cattle, dogs, cats, rabbits, aquatic mammals, etc.

As used herein, “composition” refers to a mixture of two or more compounds, elements, or molecules. In some aspects the term “composition” may be used to refer to a mixture of a nucleic acid and a delivery system.

As used herein, the terms “administration,” “administering,” and “delivering” refer to the manner in which a composition is presented to a subject. Administration can be accomplished by various art-known routes such as oral, parenteral, transdermal, inhalation, implantation, instillation, intracranial etc. Thus, an oral administration can be achieved by swallowing, chewing, sucking of an oral dosage form comprising the composition. Parenteral administration can be achieved by injecting a composition intravenously, intra-arterially, intramuscularly, intraarticularly, intrathecally, intraperitoneally, subcutaneously, etc. Injectables for such use can be prepared in conventional forms, either as a liquid solution or suspension, or in a solid form that is suitable for preparation as a solution or suspension in a liquid prior to injection, or as and emulsion. Additionally, transdermal administration can be accomplished by applying, pasting, rolling, attaching, pouring, pressing, rubbing, etc., of a transdermal composition onto a skin surface. These and additional methods of administration are well-known in the art.

As used herein, the terms “nucleotide sequence” and “nucleic acids” may be used interchangeably, and refer to DNA and RNA, as well as synthetic congeners thereof. Non-limiting examples of nucleic acids may include plasmid DNA encoding protein or inhibitory RNA producing nucleotide sequences, synthetic sequences of single or double strands, missense, antisense, nonsense, as well as on and off and rate regulatory nucleotides that control protein, peptide, and nucleic acid production. Additionally, nucleic acids may also include, without limitation, genomic DNA, cDNA, RNAi, siRNA, shRNA, mRNA, tRNA, rRNA, microRNA, hybrid sequences or synthetic or semi-synthetic sequences, and of natural or artificial origin. In one aspect, a nucleotide sequence may also include those encoding for synthesis or inhibition of a therapeutic protein. Non-limiting examples of such therapeutic proteins may include anti-cancer agents, growth factors, hypoglycemic agents, anti-angiogenic agents, bacterial antigens, viral antigens, tumor antigens or metabolic enzymes. Examples of anti-cancer agents may include interleukin-2, interleukin-4, interleukin-7, interleukin-12, interleukin-15, interferon-α, interferon-β, interferon-γ, colony stimulating factor, granulocyte-macrophage stimulating factor, anti-angiogenic agents, tumor suppressor genes, thymidine kinase, eNOS, iNOS, p53, p16, TNF-α, Fas-ligand, mutated oncogenes, tumor antigens, viral antigens or bacterial antigens. In another aspect, plasmid DNA may encode for an RNAi molecule designed to inhibit protein(s) involved in the growth or maintenance of tumor cells or other hyperproliferative cells. Furthermore, in some aspects a plasmid DNA may simultaneously encode for a therapeutic protein and one or more RNAi molecules. In other aspects a nucleic acid may also be a mixture of plasmid DNA and synthetic RNA, including sense RNA, antisense RNA, ribozymes, etc. In addition, the nucleic acid can be variable in size, ranging from oligonucleotides to chromosomes. These nucleic acids may be of human, animal, vegetable, bacterial, viral, or synthetic origin. They may be obtained by any technique known to a person skilled in the art.

As used herein, the term “peptide” may be used to refer to a natural or synthetic molecule comprising two or more amino acids linked by the carboxyl group of one amino acid to the alpha amino group of another. A peptide of the present invention is not limited by length, and thus “peptide” can include polypeptides and proteins.

As used herein, the terms “covalent” and “covalently” refer to chemical bonds whereby electrons are shared between pairs of atoms.

As used herein, the term “polymeric backbone” is used to refer to a collection of polymeric backbone molecules having a weight average molecular weight within a designated range. A polymeric backbone generally has at least two terminal ends of the molecule. In the case of a branched polymeric backbone, for example, each branch would be considered to have at least one terminal end. As such, when a molecule such as a metal chelator is described as being covalently attached to a terminal end of a polymeric backbone, it should be understood that the metal chelator is covalently attached to at least one end of the molecule where the polymeric backbone terminates. In some aspects, metal chelator molecules may be covalently attached to all terminal ends of the polymeric backbone, or to only a portion of the terminal ends.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5″ should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

It has now been unexpectedly discovered that chelating groups can be advantageously coupled to poloxamer backbones resulting in improved intra- and extracellular nucleic acid stability, thus enhancing delivery and expression. As an example, poloxamers have been shown to enhance nucleic acid delivery into living tissue. Many nucleases may limit, however, the effectiveness of such delivery through nucleic acid degradation. A nuclease is an enzyme that is capable of cleaving phosphodiester bonds of nucleotide subunits of nucleic acids. It has now been discovered that the effectiveness of gene expression in solid tissues may be enhanced through the use of a polymeric vehicle having at least one metal chelator covalently attached to a poloxamer backbone. A metal chelator functions to hinder the degradative action of a nuclease by chelating associated metal cofactors. Such a modification of the poloxamer backbone can thus inhibit nuclease activity and improve intracellular and extracellular nucleic acid stability, which in turn will result in greater transfection efficiencies.

In one aspect of the present invention, the polymeric backbone of the nucleotide delivery polymer may comprise a poloxamer. Poloxamers are generally based on an amphiphilic triblock copolymer of ethylene oxide and propylene oxide, having a central hydrophobic chain of polypropylene oxide flanked by two hydrophilic chains of polyethylene oxide. A representative general formula for poloxamer molecules is shown below.

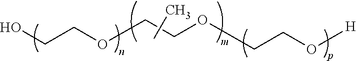

where n, m, and p are integers.

A shorthand representation of a poloxamer is HO-Pol-OH. Poloxamers improve the expression level of a reporter or a therapeutic gene, as in, for example, muscles following intramuscular injection. Without being bound to any specific theory, one hypothesis for such increased expression suggests that nucleic acid uptake may be improved via the surfactant action of poloxamers, which can thus increase cell membrane permeability by altering the structure of cell membrane lipid bilayers. Poloxamers also play a role in activating gene transcription, and thus the action of poloxymers will be mediated through various different mechanisms.

The invention includes molecules of formula I where ABC represents a “branched poloxamer.” Branched poloxamers are copolymers formed around a hub group such as glycerol, pentaerythritol, or a monosaccharide, e.g., glucose.

Because the lengths of the polymer blocks of a poloxamer backbone may vary between various polymeric constructs, many different poloxamers are considered to be within the scope of the present invention. In one aspect, for example, the average molecular weight of the poloxamer backbone may range from about 100 Da to about 100,000 Da. In another aspect, the average molecular weight of the poloxamer backbone may range from about 500 Da to about 50,000 Da. In yet another aspect, the average molecular weight of the poloxamer backbone may range from about 1000 Da to about 20,000 Da. The poloxamer backbone may also be described in terms of a ratio of ethylene oxide to propylene oxide. For example, in one aspect the ratio of ethylene oxide to propylene oxide is from about 5:1 to about 1:5. In another aspect, the ratio of ethylene oxide to propylene oxide is from about 20:1 to about 1:20.

Many poloxamers with different compositions and molecular weights are available commercially. These are frequently referred to by their trademarks or tradenames.

Suitable poloxamers include, but are not limited to, Poloxamer 101 (Pluronic® L-31), Poloxamer 105 (Pluronic® L-35), Poloxamer 108 (Pluronic® F-38), Poloxamer 123 (Pluronic® L-43), Poloxamer 124 (Pluronic® L-44), Poloxamer 181 (Pluronic® L-61), Poloxamer 182 (Pluronic® L-62), Poloxamer 184 (Pluronic® L-64), Poloxamer 185 (Pluronic® P-65), Poloxamer 188 (Pluronic® F-68), Poloxamer 217 (Pluronic® F-77), Poloxamer 231 (Pluronic® L-81), Poloxamer 234 (Pluronic® P-84), Poloxamer 235 (Pluronic® P-85), Poloxamer 237 (Pluronic® F-87), Poloxamer 238 (Pluronic® F-88), Poloxamer 282 (Pluronic® L-92), Poloxamer 288 (Pluronic® F-98), Poloxamer 331 (Pluronic® L-101), Poloxamer 333 (Pluronic® P-103), Poloxamer 334 (Pluronic® P-104), Poloxamer 335 (Pluronic® P-105), Poloxamer 338 (Pluronic® F-108), Poloxamer 401 (Pluronic® L-121), Poloxamer 403 (Pluronic® P-123), Poloxamer 407 (Pluronic® F-127), Poloxamer 183 (Calgene Nonionic® 1063-L), Poloxamer 212 (Calgene Nonionic® 1072-L), Poloxamer 215 (Calgene Nonionic® 1075-P), Poloxamer 284 (Calgene Nonionic® 1094-P), and Poloxamer 122 (Calgene Nonionic®. 1042-L). Suitable block copolymers having terminal secondary hydroxyl groups include (Meroxapals). Pluronic® 10R5, Pluronic® 17R2, Pluronic®, Pluronic® 25R2, Pluronic® 25R4, and Pluronic® 31R1. Preferred poloxamers include Pluronic® L44 [about 2.2 kDa] available from Spectrum Chemicals as Poloxamer 124.

A variety of chelators may be utilized in association with the poloxamers of the present invention to hinder the degredative action of nucleases, and any chelator capable of covalent attachment to a poloxamer backbone would be considered to be within the scope of the present invention. Additionally, in one aspect of the present invention, metal chelators are capable of chelating metals such as Fe²⁺, Fe³⁺, Mg²⁺, Zn²⁺, Mn²⁺, Cu²⁺, Ca⁺², Ni⁺², Li⁺, Na⁺, K⁺, and La⁺³. Non-limiting examples of metal chelators may include cyclic metal chelators or open chain metal chelators. In one aspect, a cyclic metal chelator may include, without limitation, crown ethers, benzocrown ethers, cryptands, benzocryptands, and combinations thereof.

Examples of suitable crown ethers for use herein include 12-crown-4 [1, 4, 7, 10-tetraoxacyclododecane]; 15-crown-5 [1, 4, 7, 10, 13-pentaoxacyclopentadecane]; 18-crown-6 [1, 4, 7, 10, 13, 16-hexaoxacyclooctadecane]; 21-crown-7 [1, 4, 7, 10, 13, 16, 19-heptaoxacycloheneicosane]; 24-crown-8 [1, 4, 7, 10, 13, 16, 19, 22-octaoxacyclotetracosane], their mono- and poly-aza- and thia-analogs; benzo-fused derivatives of such oxa- and hetero-crown ethers, and cryptands, such as (2,2,2) cryptand [1, 10-diaza-4, 7, 13, 16, 21, 24-hexaoxa-bicyclo[8,8,8]hexacosane], other cryptands with different cavity size [such as (1,2,2), (2,2,3), (2,3,3) cryptands], and their mono- and poly-aza- and thia-analogs, as well as benzo-fused derivatives.

Examples of suitable chelators are ethylenediaminetetraacetic acid, diethylenetriaminepentaacetic acid, and nitrilotriacetic acid. Still other examples of suitable chelators are diethylenetriamine [1, 4, 7-triazaheptane], triethylenetetramine [1, 4, 7, 10-tetraazadecane], tetraethylenepentamine [1, 4, 7, 10, 13-pentaazatridecane], pentaethylenehexamine [1, 4, 7, 10, 13, 16-hexaazahexadecane].

Preferred open chain metal chelators may include, without limitation, EDTA, DTPA, short polyamines, or combinations thereof. It should be noted that many chelators such as crown ethers have not been previously considered for use in biological systems due to their known toxic effects. It has now been discovered that such previously toxic chelators can be safely used to hinder nuclease activity in biological systems when coupled to a poloxamer backbone. Additionally, in one aspect a metal chelator may be included in the compositions of the present invention as a coformulant, and thus would not be covalently attached to the poloxamer backbone. Thus in one specific aspect it is contemplated that a noncovalently bound metal chelator may be formulated with poloxamer backbone having additional metal chelator covalently bound. In another specific aspect, a noncovalently bound metal chelator may be formulated with poloxamer backbone that does not have additional metal chelator covalently bound.

The point of attachment of the chelator to the poloxamer backbone can vary widely depending on the chelator, the nature of the backbone, the intended uses of the delivery vehicle, etc. In one aspect, for example, the point of attachment may include a nitrogen atom from the chelator or a tether molecule, either present in the ligand itself, like a carboxyl group in EDTA or DTPA, or specially attached as a functionalized “tail”.

As disclosed above, cationic moieties can be covalently attached to poloxamers to modulate the affinity of the poloxamers for nucleic acids, and/or to retard nucleic acid digestion by endonucleases through partial condensation of the nucleic acids. Representative short polyamines include tren, tetren, pentren, spermidine and spermine. These amines are capable of chelating metal cations, and as such may be utilized to ligate metal ions in metalloprotease enzymes in addition to those properties described above. Cationic poloxamers could also lead to enhanced gene transfer by their attraction to, and crossing of, the relatively negatively charged cell membrane, thus facilitating nucleic acid uptake.

In another aspect, the present invention additionally provides nucleotide delivery compositions. Such a composition may include a nucleotide sequence and a poloxamer backbone having a metal chelator covalently coupled to one or more terminal end(s) of the poloxamer backbone, wherein the nucleotide sequence is associated with the poloxamer backbone.

Any known nucleic acid may be utilized in the compositions and methods according to aspects of the present invention, and as such, the nucleic acids described herein should not be seen as limiting. General examples of nucleotide sequences may include DNA, cDNA, RNA, siRNA, RNAi, shRNA, mRNA, microRNA, etc. In one aspect, for example, the nucleic acid may include a plasmid encoding for a protein, polypeptide, or peptide. Numerous peptides are well known that would prove beneficial when formulated as pharmaceutical compositions according to aspects of the present invention. Non-limiting examples of a few of such peptides may include interleukin-2, interleukin-4, interleukin-7, interleukin-12, interleukin-15, interferon-α, interferon-β, interferon-γ, colony stimulating factor, granulocyte-macrophage colony stimulating factor, angiogenic agents, clotting factors, hypoglycemic agents, apoptosis factors, anti-angiogenic agents, thymidine kinase, p53, IP10, p16, TNF-α, Fas-ligand, tumor antigens, neuropeptides, viral antigens, bacterial antigens, and combinations thereof. In one specific aspect, the nucleic acid may be a plasmid encoding for interleukin-12. In another aspect, the nucleic acid may be a plasmid encoding for an inhibitory ribonucleic acid. In yet another aspect, the nucleic acid may be a synthetic short interfering ribonucleic acid. In a further aspect, the nucleic acid is an anti-sense molecule designed to inhibit expression of a therapeutic peptide.

In another aspect, the present invention additionally provides nucleotide delivery compositions. Such compositions include a nucleotide sequence pre-complexed with one or more of a cationic delivery system such as but not limited to a cationic polymer, cationic lipid, or cationic peptide and compound of the invention.

It is also contemplated that a filler excipient may be included in pharmaceutical compositions according to certain aspects of the present invention. Such filler may provide a variety of beneficial properties to the formulation, such as cryoprotection during lyophilization and reconstitution, binding, isotonic balance, stabilization, etc. It should be understood that the filler material may vary between compositions, and the particular filler used should not be seen as limiting. In one aspect, for example, the filler excipient may include various sugars, sugar alcohols, starches, celluloses, and combinations thereof. In another aspect, the filler excipient may include lactose, sucrose, trehalose, dextrose, galactose, mannitol, maltitol, maltose, sorbitol, xylitol, mannose, glucose, fructose, polyvinyl pyrrolidone, glycine, maltodextrin, hydroxymethyl starch, gelatin, sorbitol, ficol, sodium chloride, calcium phosphate, calcium carbonate, polyethylene glycol, and combinations thereof. In yet another aspect the filler excipient may include lactose, sucrose, trehalose, dextrose, galactose, mannitol, maltitol, maltose, sorbitol, xylitol, mannose, glucose, fructose, polyvinyl pyrrolidone, glycine, maltodextrin, and combinations thereof. In one specific aspect, the filler excipient may include sucrose. In another specific aspect, the filler excipient may include lactose.

In some aspects it may be beneficial to functionalize the poloxamer to allow targeting of specific cells or tissues in a subject or culture. Such targeting is well known, and the examples described herein should not be seen as limiting. In one aspect, for example, the poloxamer may include a targeting moiety covalently attached to the backbone or the chelator. Examples of such targeting moieties may include transferrin, asialoglycoprotein, antibodies, antibody fragments, low density lipoproteins, cell receptors, growth factor receptors, cytokine receptors, folate, transferrin, insulin, asialoorosomucoid, mannose-6-phosphate, mannose, interleukins, GM-CSF, G-CSF, M-CSF, stem cell factors, erythropoietin, epidermal growth factor (EGF), insulin, asialoorosomucoid, mannose-6-phosphate, mannose, Lewis^{X} and sialyl Lewis^{X}, N-acetyllactosamine, folate, galactose, lactose, and thrombomodulin, fusogenic agents such as polymixin B and hemaglutinin HA2, lysosomotrophic agents, nucleus localization signals (NLS) such as T-antigen, and combinations thereof. The selection and attachment of a particular targeting moiety is well within the knowledge of one of ordinary skill in the art.

The present invention also provides lyophilized pharmaceutical compositions that may be stored for periods of time and reconstituted prior to use. In one aspect, for example, a lyophilized pharmaceutical composition may include a lyophilized mixture of a filler excipient, a nucleic acid, and a poloxamer. Lyophilized pharmaceutical compositions may be in a variety of forms, ranging from dry powders to partially reconstituted mixtures.

The present invention additionally provides methods for enhancing expression of a nucleotide sequence in a solid tissue of a subject. Such a method may include mixing the nucleotide sequence with a nucleotide sequence delivery polymer to form a gene delivery composition, where the nucleotide sequence delivery polymer further includes a poloxamer backbone having a metal chelator covalently coupled to at least one terminal end of the poloxamer backbone. The method may further include delivering the gene delivery composition into the solid tissue of the subject. The metal chelator may be covalently coupled to one terminal end or to both terminal ends of the poloxamer backbone. The gene delivery composition may be delivered into any solid tissue or subset of tissue to achieve a therapeutic result. Non-limiting examples of such solid tissues may include solid tumors, muscle tissue, fat tissue, connective tissue, joint tissue, neural tissue, organ tissue, bone tissue, skin tissue, etc. Additionally, it is contemplated that the compositions according to aspects of the present invention may be delivered to body cavities, both dorsal and ventral, including, for example, cranial, orbital, peritoneal, pelvic, pericardial, intravaginal, etc.

Aspects of the present invention also provide methods of using pharmaceutical compositions for transfection of a variety of cells. For example, in one aspect transfecting a mammalian cell may include contacting the mammalian cell with a composition as described herein, and incubating the mammalian cell under conditions to allow the composition to enter the cell and elicit biological activity of the nucleotide sequence. Such transfection techniques are known to those of ordinary skill in the art.

### EXAMPLES

The following examples are provided to promote a more clear understanding of certain embodiments of the present invention, and are in no way meant as a limitation thereon.

### Example 1

### Synthesis of Chelator-Linked Poloxamers: Pentetic Acid-Linked Poloxamer

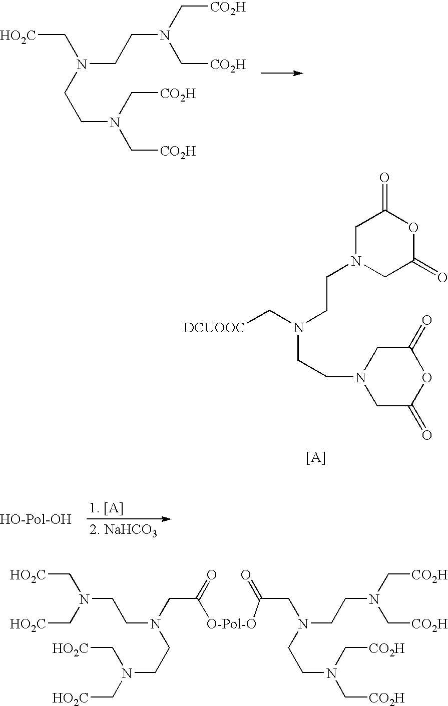

Diethylenetriaminepentaacetic acid (1 g, 2.5 mmol) was dissolved in 20 ml of dry DMSO. Dicyclohexylcarbodiimide (1.34 g, 6.5 mmol) was added, and the reaction mixture was stirred overnight. Dicyclohexylurea was removed by filtration, and poloxamer 124 (1 g, 450 μmol) was added to the filtrates. The reaction mixture was allowed to stand for 1 week; the resulting solution was treated with 30 ml of 10% aq. NaHCO₃ to open the cyclic anhydrides. After 4 hrs, the mixture was further diluted with water to 120 ml and then dialyzed (membrane cutoff 1000 Da) against distilled water. The concentration of dialyzate afforded pentetic acid-poloxamer conjugate [1 g, after mechanical losses] as a glassy material.

### Example 2

### Synthesis of Aza-Crown-Linked Poloxamer

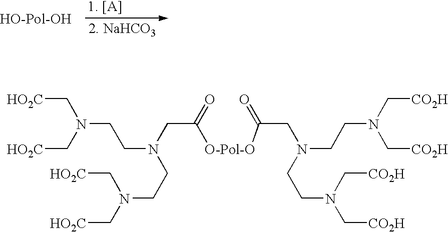

An aza-crown-linked poloxamer is constructed as follows. Poloxamer 124 (500 mg, 220 μmol) was dissolved in toluene (3 ml), and the resulting solution was treated with 2 ml (4 mmol) of 2M phosgene solution in toluene. After 3 hrs at room temperature, the mixture was concentrated in vacuum, the residue was re-dissolved in 3 ml toluene and concentrated again. The residue was dissolved in dry chloroform (5 ml). To this solution was added aza-18-crown-6 [1-aza-4, 7, 10, 13, 16-pentaoxacyclooctadecane (125 mg, 500 μmol) and Hunig's base (100 μl, 574 μmol). After 70 hrs the reaction mixture was concentrated in vacuum, the residue was re-dissolved in distilled water and dialyzed [membrane cutoff 1000 Da] against distilled water. Concentration of the dialyzate afforded 410 mg of the title compound.

Proton NMR (D₂O): 4.20 ppm (t, CH₂OC═O); 3.7-3.5 ppm [(—CH₂—CH₂—O—), both crown and poloxamer)]; 3.4 ppm (m, crown CH₂N); 1.1 ppm (m, poloxamer —(CH₃)CH-CH₂—).

### Example 3

### Synthesis of Poloxamer Linked with Cationic Chelator

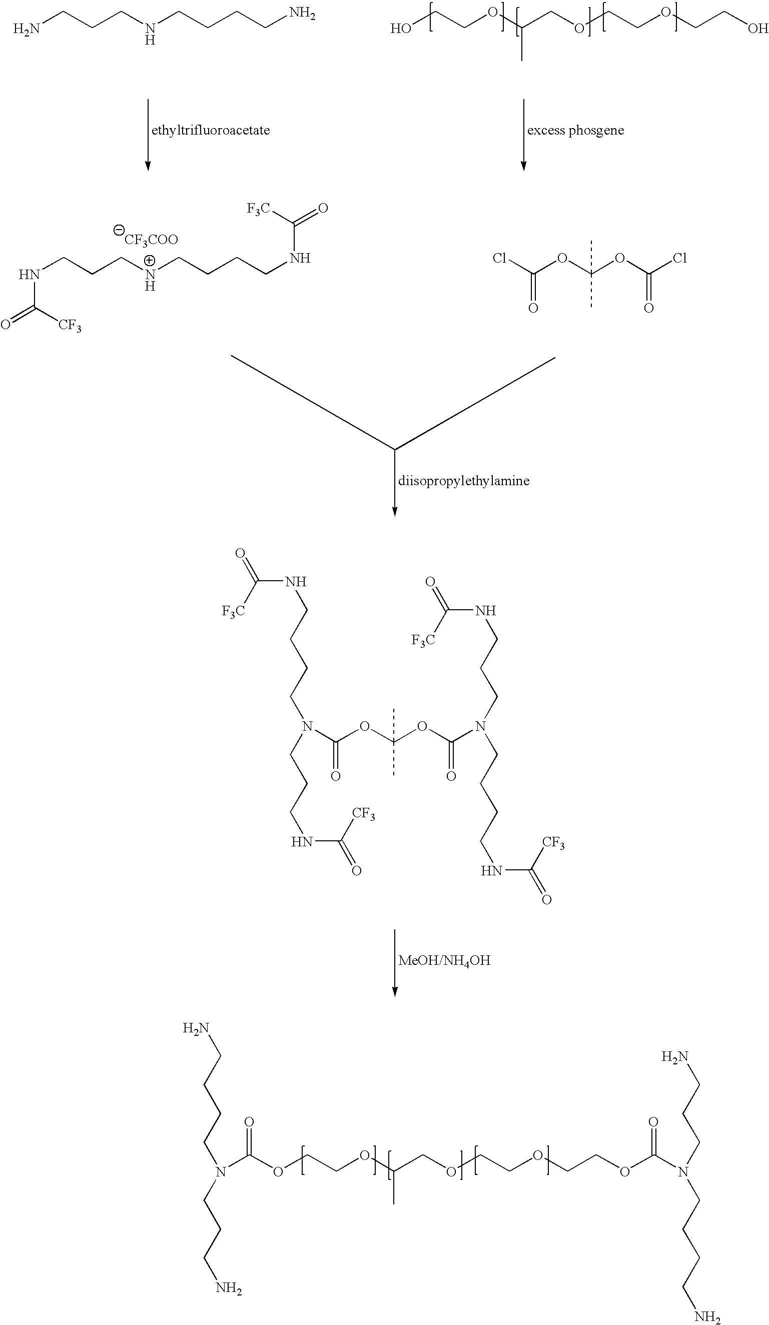

Cationic chelator-linked poloxamers were constructed as follows: Three grams of Poloxamer 124 was placed in a 50 mL round bottomed flask and heated with stirring under high vacuum at 80° C. for 5 hours to remove water. The poloxamer was dissolved in 2 ml of toluene and 4 ml of 2M phosgene (in toluene) were added. The solution was cooled to 0° C. for 5 min, after which it was allowed to warm to room temperature. The reaction was allowed to proceed with stirring for 5 h at room temperature, after which toluene was removed to leave a clear viscous liquid. The bischloroformate-activated poloxamer was stored under argon at −20° C. until further use.

Proton NMR (D₂O): 1.2 ppm (m, (—O—CH₂—CH(CH₃)—), 3.3 ppm (m, (—O—CH₂—CH(CH₃)—), 3.4 ppm (m, (—O—CH₂—CH(CH₃)—), 3.6 ppm (t, (—O—CH₂—CH₂—), 3.8 ppm (t, Cl—C(O)—O—CH₂—CH₂—), 4.5 ppm (t, Cl—C(O)—O—CH₂—CH₂—).

The two primary amines of spermidine were protected in the presence of the secondary amine using a procedure adapted from O'Sullivan, Tet. Lett. (1995), 36, 3451. Two grams of spermidine were placed in a 100 ml round bottomed flask and dissolved in 25 ml of acetonitrile. Ethyltrifluoroacetate (6.8 g) was added, followed by 0.3 g of water. The clear solution was refluxed overnight (18 h), after which the solvents were evaporated under vacuum to give a waxy solid material. To purify the product, 25 ml of ethyl acetate was added, giving a cloudy mixture. The solution was filtered through a glass fritted funnel (10-15 micron) to remove the insoluble impurities, and the clear solution was dried to give a white powder (5.47 g).

Proton NMR: 1.5 ppm (m, F₃C—C(O)—NH—CH₂—CH₂—CH₂—NH—CH₂—CH₂—CH₂—CH₂—NH—C(O)—CF₃), 1.6 ppm (m, F₃C—C(O)—NH—CH₂—CH₂—CH₂—NH—CH₂—CH₂—CH₂—CH₂—NH—C(O)—CF₃), 1.8 ppm (m, F₃C—C(O)—NH—CH₂—CH₂—CH₂—NH—CH₂—CH₂—CH₂—CH₂—NH—C(O)—CF₃), 3.0 (m, F₃C—C(O)—NH—CH₂—CH₂—CH₂—NH—CH₂—CH₂—CH₂—CH₂—NH—C(O)—CF₃), 3.3 ppm (t, F₃C—C(O)—NH—CH₂—CH₂—CH₂—NH—CH₂—CH₂—CH₂—CH₂—NH—C(O)—CF₃), 3.4 ppm (t, F₃C—C(O)—NH—CH₂—CH₂—CH₂—NH—CH₂—CH₂—CH₂—CH₂—NH—C(O)—CF₃).

The activated poloxamer from above was functionalized with the TFE-protected spermidine in the following manner. Three grams of poloxamer 124 bischloroformate were dissolved in 4 ml of freshly distilled THF, giving a clear solution. Solid protected spermidine (1.0 g) was added which resulted in a slightly yellow cloudy mixture. Diisopropylethylamine (1.5 ml) was added and the mixture immediately became a clear yellow homogeneous solution. The reaction was allowed to proceed at room temperature with stirring for 24 h. The THF was removed under vacuum to give a slightly yellow viscous liquid (3.8 g). The TFE-protection was removed from the spermidine groups in the following manner. The viscous functionalized poloxamer from above was dissolved in 30 ml of a 2:1 mixture of methanol and ammonium hydroxide. The solution was heated to reflux overnight (18 h). After the methanol was removed under vacuum, the purified, bis-spermidine poloxamer was obtained by dialysis against pure water using a SpectraPor 7 (MWCO 1000) dialysis bag. The dialysis was performed over 48 h, with bath a bath change every 8 h. The pure material was obtained after freeze drying the dialysate (3.2 g).

### Example 4

### Nucleic Acids Formulation with Modified Poloxamers

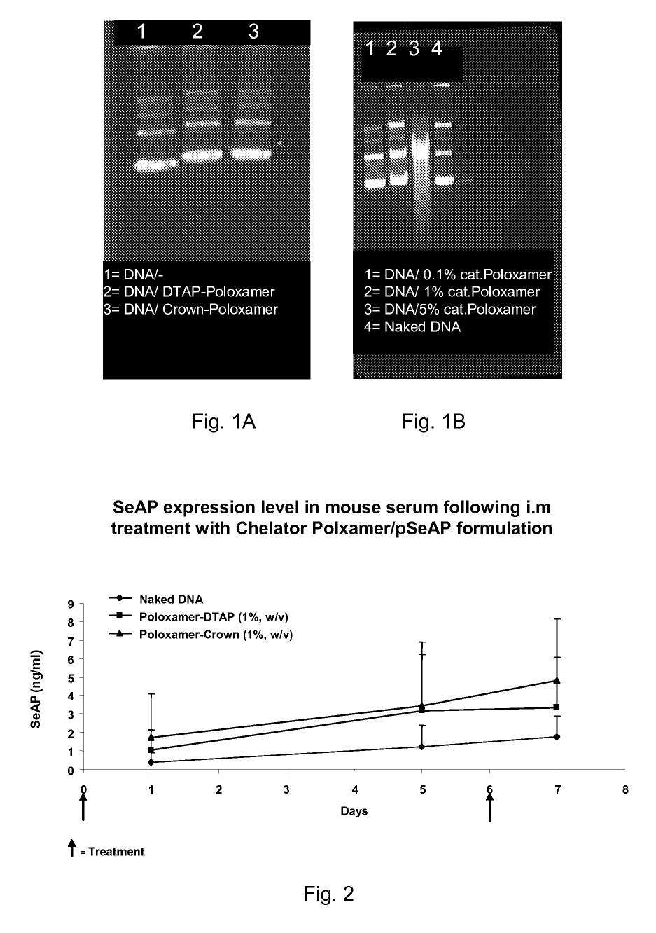

Modified poloxamers are gently mixed with 1 mg/ml of nucleic acids in water or saline solution (0.15 M) at variable concentrations. Formulated polymer (5%)/plasmid solutions are analyzed by gel electrophoresis in order to verify interaction between formulated plasmid and the modified poloxamers. Comparison between unformulated plasmid DNA and DNA formulated with divalent cation chelators have similar movement though the gel and therefore indicate no binding between plasmid DNA and the chelator modified poloxamers (FIG. 1A). Additionally, cationic poloxamers are able to condense naked plasmid DNA at polymer concentrations above 1% (FIG. 1B).

Furthermore, a reduction in the particle size of cationic poloxamers formulated with plasmid is observed in comparison to unformulated plasmid (Table 1). This reduction in size may be indicative of complexation of the DNA by the cationic poloxamers.

TABLE 1

| | Particle size (nm) | Polydispersity |
|---|---|---|
| Naked DNA (1 mg/ml) | 494.6 | 0.347 |
| Cationic Poloxamer 1% | 133.1 | 0.165 |
| (w/v)/pSeAP 1 mg/ml | | |

### Example 5

### Gene Transfer into Skeletal Muscle by Crown Poloxamers

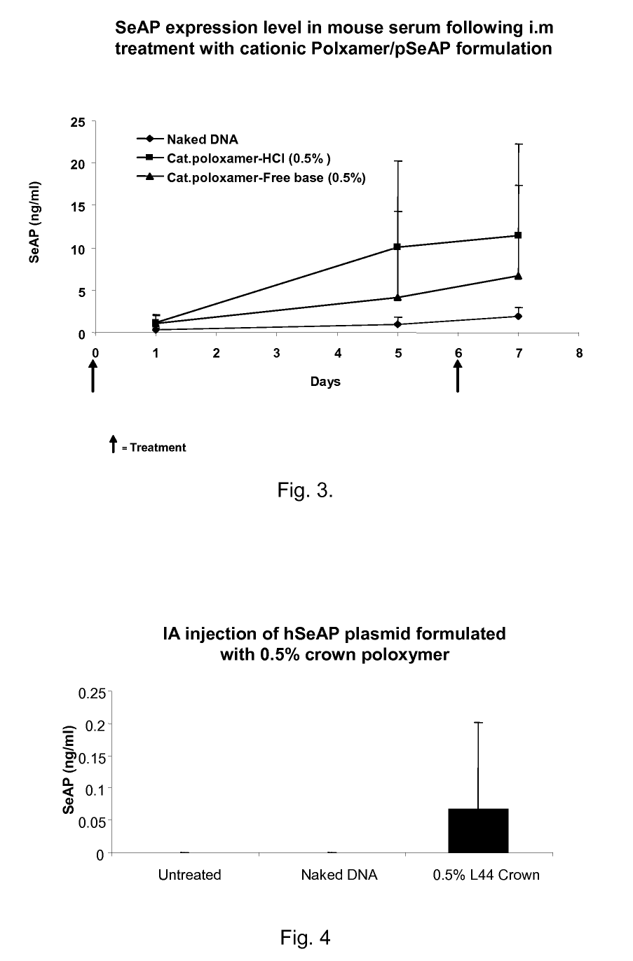

Female ICR mice (12 weeks, 27-50 grams) are treated twice, once at time zero and once at day six, with an intramuscular injection into each tibialis muscle (left and right hindlimbs) of 25 μg of human secreted alkaline phosphatase (hSEAP) expression plasmid formulated with neutral or cationic chelating poloxamers. Serum is collected retro-ortibally at various times after treatments for the determination of reporter gene expression level. As can be seen in FIGS. 2 and 3, both neutral and cationic chelating poloxamers show an enhancement in SeAP expression levels in comparison to the naked plasmid DNA group.

### Example 6

### Gene Transfer into Knee Joint by Crown Poloxamer

A plasmid encoding the hSeAP reporter gene is formulated with the crown poloxamer of Example 2 at 0.5%. The final DNA concentration is at 1.0 mg/ml. A total volume of 25 μl is injected into the left and right knees of female ICR mice (12 weeks, 27-50 grams). At 24 hours after the injection, serum is obtained from the animals via retro-orbital puncture. The hSeAP levels are determined using a commercially available calorimetric assay. The results show intra-articular injection of unformulated “naked” DNA does not produce detectable expression levels, whereas injection of formulated hSeAP plasmid is capable of producing sufficiently high expression for systemic detection from a single injection (FIG. 4).

### Example 7

### Gene Transfer into Solid Tumors by Crown Poloxamers

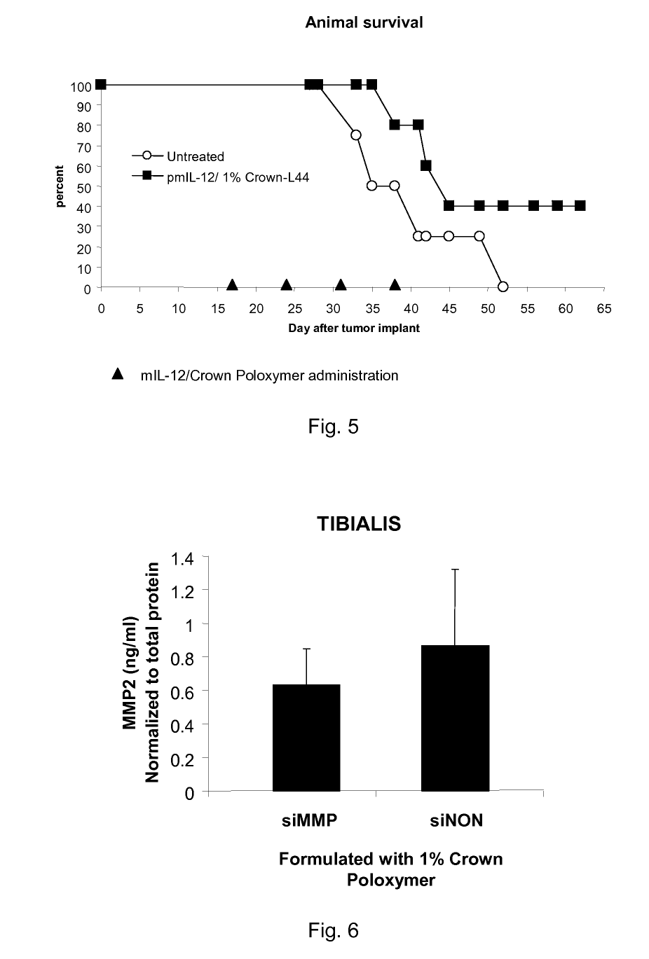

Tumors are implanted in mice by administration of 5×10⁵ murine squamous cell carcinoma VII (SCCVII) cells into the flank of syngeneic CH3 mice. Tumors are allowed grow until they reached a volume of ˜80 mm³. At 17 days after injection, tumors were injected with 30 μl of mouse IL-12 plasmid (pmIL-12) formulated with crown poloxamer at 1%. The final DNA concentration is 1.0 mg/ml. The tumors are repeatedly injected (weekly) for a total of 4 treatments. The results are shown in FIG. 5.

### Example 8

### siRNA Delivery and Gene Knockdown in Solid Tissues by Crown Poloxamers

The ability to knock-down endogenous gene expression in muscle using siRNA formulated with crown poloxamer is evaluated. Left and Right tibialis muscle of ICR mice are injected twice over three days with 25 μl of formulated siRNA targeting matrix metalloprotease 2 (MMP2). The siRNA is formulated with 1% crown poloxamer at a final RNA concentration of 1.0 mg/ml. One day following the second injection, the mice are euthanized and tibialis muscles are harvested for MMP2 protein analysis. MMP2 protein levels are determined using a commercially available ELISA assay. Compared to the non-silencing control, administration of formulated MMP2 siRNA results in a 28% knockdown in protein expression (FIG. 6). These results suggest that crown poloxamer may be utilized for delivery of siRNAs to muscle and other solid tissues.

### Example 9

### Gene Transfer into Ischemic Cardiac Tissue by Crown Poloxamers to Promote Vascularization and Restore Cardiac Function

Female ICR mice are anesthetized with isofluorane. Approximately 40 μl of plasmid encoding for vascular endothelial growth factor (VEGF) formulated with neutral or cationic chelating poloxamers is injected percutaneously into the left ventricular wall using a syringe with a 27G needle. In some cases it may be necessary to perform the injection under the guidance of echocardiography. At various times after injection, hearts are harvested and analyzed for VEGF expression levels.

### Example 10

### siRNA Delivery and Gene Knockdown of Matrix Metalloproteases to Inhibit Tumor Metastases by Crown Poloxamers

Tumors are implanted in mice by administration of 5×10⁵ B16BL6 mouse melanoma into the flank of syngeneic C57BL/6 mice. Tumors are allowed to grow until reaching a volume of ˜80 mm³, at which point they are injected with 25 ml of formulated siRNA targeting matrix metalloprotease 2 (MMP2). The siRNA is formulated with 1% crown poloxamer at a final RNA concentration of 1.0 mg/ml. Twice weekly injections are performed for the next three weeks. One day after the last injection tumors are harvested and analyzed for MMP2 protein. As a measure of tumor metastasis, the animals lungs are harvested and tumor modules in the lungs are counted.

### Example 11

### Co-Formulation of Crown Poloxamer with Cationic Polymer Particles for Gene Transfer

Tumors are implanted in mice by administration of 5×10⁵ murine squamous cell carcinoma VII (SCCVII) cells into the flank of syngeneic CH3 mice. Tumors are allowed grow until they reached a volume of 50-80 mm³. Cationic polymeric particles are prepared by mixing plasmid DNA encoding for a therapeutic gene and a cationic polymer such as branched polyethyleneimine at a 1:1 volume such that the final nitrogen/phosphate ration is in a range of 1/1 to 20/1. The formulation is incubated for 15 minutes at room temperature to allow the complexes to form. The cationic particle mixture is then mixed with a crown poloxamer solution to achieve a final poloxamer concentration of 0.25 to 1.5%. Tumors are injected with this solution and at various time points after administration tumors are harvested for quantification of protein corresponding the delivered gene.

### Example 12

### Co-Formulation of Crown Poloxamer with Cationic Lipid Particles for siRNA Delivery into Solid Tumors

The cationic particle Tumors are implanted in mice by administration of 5×10⁵ murine squamous cell carcinoma VII (SCCVII) cells into the flank of syngeneic CH3 mice. Tumors are allowed grow until they reached a volume of 50-80 mm³. Cationic liposomes are prepared and diluted to 1.9 mg/ml in 5% dextrose. The siRNA molecules are diluted to 0.3 mg/ml in 5% dextrose. Equal volumes of the 2 reagents are mixed together and the solution is incubated for 15 minutes at room temperature to allow the complexes to form. The cationic particle mixture is then mixed with a crown poloxamer solution to achieve a final poloxamer concentration of 0.25 to 1.5%. Tumors are injected with this solution and at various time points after administration the tumors are harvested for quantification of transcript that was targeted by the siRNA.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A compound of the formula:
R^{A}-O-A-B-C-R^{C}
or a pharmaceutically acceptable salt thereof, wherein
A is (—C₂H₄—O—)₂₋₁₄₁;
B is (—C₃H₆—O—)₁₆₋₆₇;
C is (—C₂H₄—O—)₂₋₁₄₁;
R^{A} and R^{C} are the same or different, and are R′-L- or H, wherein at least one of R^{A} and R^{C} is R′-L-;
L is a bond, —CO—, —CH₂—O—, or —O—CO—;
R′ is a metal chelator, wherein the metal chelator is
(a) R^{N}NH—;
(b) R^{N}₂N—;
(c) (R″—(N(R″)—CH₂CH₂)ₓ)₂—N—CH₂CO—;
(d) a crown ether selected from the group consisting of
12-crown-4,
15-crown-5,
18-crown-6,
20-crown-6,
21-crown-7, or
24-crown-8;
(e) a substituted-crown ether, wherein the substituted-crown ether has
(1) one or more of the crown ether oxygens independently replaced by NH or S,
(2) one or more of the crown ether —CH₂—CH₂— moieties replaced by —C₆H₄—, —C₁₀H₆—, or —C₆H₁₀—,
(3) one or more of the crown ether —CH₂—O—CH₂—moieties replaced by —C₄H₂O— or —C₅H₃N—, or
(4) any combination thereof;
(f) a cryptand, wherein the cryptand is selected from the group consisting of
(1,2,2) cryptand,
(2,2,2) cryptand,
(2,2,3) cryptand, or
(2,3,3) cryptand;
(g) a substituted-cryptand, wherein the substituted-cryptand has
(1) one or more of the cryptand ether oxygens independently replaced by NH or S,
(2) one or more of the crown ether —CH₂—CH₂— moieties replaced by —C₆H₄—, —C₁₀H₆—, or —C₆H₁₀—,
(3) one or more of the crown ether —CH₂—O—CH₂—moieties replaced by —C₄H₂O— or —C₅H₃N—, or
(4) any combination thereof;
each R^{N} is independently H-(R^{D})₁₋₅, wherein each R^{D} is independently —NH(CH₂CH₂)—, —NH(CH₂CH₂CH₂)—, or —NH(CH₂CH₂CH₂CH₂)—;
each x is independently 0-2;
and R″ is HO₂C—CH₂—.

2. A compound according to claim 1, wherein each R is the same or different and is R′-L-.

3. A compound according to claim 1, wherein at least one metal chelator is a member selected from the group consisting of crown ether, substituted-crown ether, ether, cryptand, or substituted-cryptand, wherein one of more of the metal chelator oxygens may be independently replaced by NH or S.

4. A compound according to claim 3, wherein at least one metal chelator is selected from the group consisting of crown ethers, substituted-crown ethers, cryptands, substituted-cryptands.

5. A compound according to claim 4, wherein at least one metal chelator is a crown ether.

6. The nucleotide delivery polymer of claim 1, wherein at least one metal chelator is selected from the group consisting of (a) R^{N}NH—;
(b) R^{N}₂N—; and
(c) (R″—(N(R″)—CH₂CH₂)ₓ)₂—N—CH₂CO—.

7. A compound according to claim 6, wherein at least one metal chelator is (R″—(N(R″)—CH₂CH₂)ₓ)₂—N—CH₂CO—.

8. A compound according to claim 6, wherein at least one metal chelator is selected from the group consisting of R^{N}NH— and R^{N}₂N—.

9. A compound according to claim 1 which is

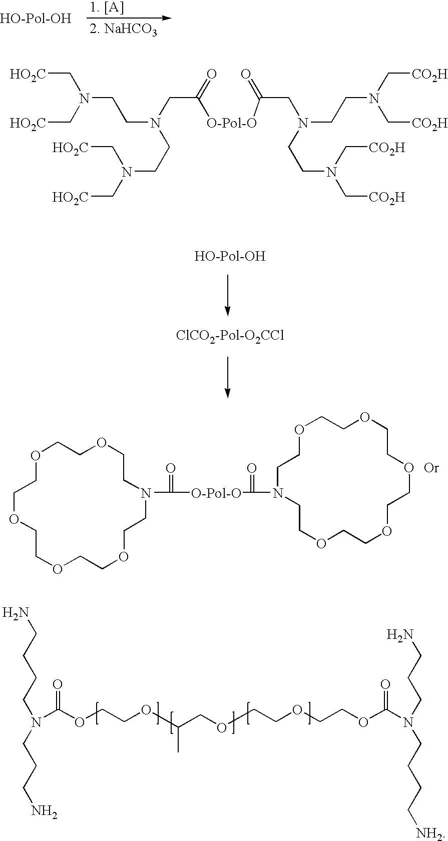

10. A gene delivery composition, comprising:
a nucleotide sequence; and
a compound of the formula:
R^{A}—O-A-B-C-R^{C}
or a pharmaceutically acceptable salt thereof, wherein:
A is (—C₂H₄—O—)₁₂₋₁₄₁;
B is (—C₃H₆—O—)₂₀₋₅₆;
C is (—C₂H₄—O—)₁₂₋₁₄₁;
R^{A} and R^{C} are the same or different, and are R′-L- or H, wherein at least one of R^{A} and R^{C} is R′-L-;
L is a bond, —CO—, —CH₂—O—, or —O—CO—;
R′ is a metal chelator, wherein the metal chelator is
(a) R^{N}NH—;
(b) RN₂N—;
(c) (R″—(N(R″)—CH₂CH₂)ₓ)₂—N—CH₂CO—;
(d) a crown ether selected from the group consisting of
12-crown-4,
15-crown-5,
18-crown-6,
20-crown-6,
21-crown-7, or
24-crown-8;
(e) a substituted-crown ether, wherein the substituted-crown ether has
(1) one or more of the crown ether oxygens independently replaced by NH or S,
(2) one or more of the crown ether —CH₂—CH₂— moieties replaced by —C₆H₄—, —C₁₀H₆—, or —C₆H₁₀—,
(3) one or more of the crown ether —CH₂—O—CH₂—moieties replaced by —C₄H₂O— or —C₅H₃N—, or
(4) any combination thereof;
(f) a cryptand, wherein the cryptand is selected from the group consisting of
(1,2,2) cryptand,
(2,2,2) cryptand,
(2,2,3) cryptand, or
(2,3,3) cryptand;
(g) a substituted-cryptand, wherein the substituted-cryptand has
(1) one or more of the cryptand ether oxygens independently replaced by NH or S,
(2) one or more of the crown ether —CH₂—CH₂— moieties replaced by —C₆H₄—, —C₁₀H₆—, or —C₆H₁₀—,
(3) one or more of the crown ether —CH₂—O—CH₂—moieties replaced by —C₄H₂O— or —C₅H₃N—, or
(4) any combination thereof;
each R^{N} is independently H—(R^{D})₁₋₅, wherein each R^{D} is independently —NH(CH₂CH₂)—, —NH(CH₂CH₂CH₂)—, or —NH(CH₂CH₂CH₂CH₂)—;
each x is independently 0-2;
and R″ is HO₂C—CH₂—.

11. The composition of claim 10, wherein the nucleotide sequence includes a member selected from the group consisting of DNA, cDNA, RNA, siRNA, RNAi, shRNA, mRNA, microRNA, and combinations thereof.

12. The composition of claim 10, wherein the nucleotide sequence is a plasmid encoding for a member selected from the group consisting of RNAi, siRNA, shRNA, mRNA, microRNA, and combinations thereof.

13. The composition of claim 10, wherein the nucleotide sequence is a plasmid encoding for a peptide.

14. The composition of claim 10, wherein the nucleotide sequence is a plasmid encoding for a member selected from the group consisting of interleukin-2, interleukin-4, interleukin-7, interleukin-12, interleukin-15, interferon-α, interferon-β, interferon-γ, colony stimulating factor, granulocyte-macrophage colony stimulating factor, angiogenic agents, clotting factors, hypoglycemic agents, apoptosis factors, anti-angiogenic agents, thymidine kinase, p53, IP10, p16, TNF-α, Fas-ligand, tumor antigens, neuropeptides, viral antigens, bacterial antigens, and combinations thereof.

15. The composition of claim 10, wherein the nucleotide sequence is an anti-sense molecule configured to inhibit expression of a therapeutic peptide.

16. The composition of claim 10, wherein at least one metal chelator is selected from the group consisting of crown ethers, substituted-crown ethers, cryptands, and substituted-cryptands.

17. The composition of claim 10, wherein at least one metal chelator is (R″—(N(R″)—CH₂CH₂)ₓ)₂—N—CH₂CO—.

18. The composition of claim 10, wherein at least one metal chelator is selected from the group consisting of R^{N}NH— and R^{N}₂N—.

19. A gene delivery composition comprising a condensed nucleic acid and a compound of claim 1, wherein the nucleic acid is fully condensed with a condensing molecule into 50-300 nm size particles.

20. The gene delivery composition of claim 18 where the condensing molecule is preferably a cationic polymer, a cationic lipid or a cationic peptide.

21. A method of enhancing delivery and/or expression of a sequence in a solid tissue of a subject, comprising delivering a composition of claim 10 into the solid tissue of the subject.

22. The method of claim 21, wherein the solid tissue includes a member selected from the group consisting of solid tumors, muscle tissue, fat tissue, connective tissue, joint tissue, neural tissue, organ tissue, bone tissue, skin tissue, and combinations thereof.

23. A method of enhancing delivery and/or expression of a nucleotide sequence in a solid tissue of a subject, comprising:
mixing the nucleotide sequence with a nucleotide delivery polymer to form a nucleotide delivery composition, the nucleotide delivery polymer further comprising a poloxamer backbone having a metal chelator covalently coupled to at least one terminal end of the poloxamer backbone; and
delivering the nucleotide delivery composition into the solid tissue of the subject.

24. The method of claim 23 wherein the metal chelator is covalently coupled to both terminal ends of the poloxamer backbone.

25. The method of claim 23, wherein the solid tissue includes a member selected from the group consisting of solid tumors, muscle tissue, fat tissue, connective tissue, joint tissue, neural tissue, organ tissue, bone tissue, skin tissue, and combinations thereof.

26. A gene delivery composition, comprising:
a nucleotide sequence;
a poloxamer backbone; and
a metal chelator.

27. A method of enhancing delivery and/or expression of a nucleotide sequence in at least one body cavity of a mammal, comprising delivering a composition of claim 10 into a body cavity of the mammal.

28. The method of claim 27, wherein body cavity is a Ventral body cavity, thoracic cavity, abdominal cavity, pelvic cavity, dorsal cavity, cranial cavity, spinal cavity, or a combination thereof
